(19) United States
(12) Patent Application Publication
(10) Pub. No.: US 2012/0090622 A1
(43) Pub. Date: Apr. 19, 2012

(54) BREATHING ASSISTANCE APPARATUS WITH TWO NASAL PRONGS

(75) Inventor: Eric Chang, Taichung Hsien (TW)

(73) Assignee: HSINER CO., LTD., Taichung Hsien (TW)

(21) Appl. No.: 12/943,259

(22) Filed: Nov. 10, 2010

(30) Foreign Application Priority Data
Oct. 13, 2010 (TW) 099134882

(51) Int. Cl. A61M 16/04

(52) U.S. Cl. 128/207.18

(57) ABSTRACT

A breathing assistance apparatus includes a nasal mask unit, an insert plate, two nasal prongs, a head strap unit, and an intake tube. The nasal mask unit includes a rear wall having an inner surrounding surface defining a prong-receiving hole, a forwardly converging front wall formed with a tube-connecting hole, an insert groove formed in the inner surrounding surface, and two side straps extending respectively from two opposite sides of the rear wall. The front and rear walls define a compartment in fluid communication with the tube-connecting hole and the prong-receiving hole. The insert plate is inserted into the insert groove to thereby be positioned in the prong-receiving hole. Each of the nasal prongs has a nose-engaging end portion, a plate-engaging end portion fitted within the corresponding mounting hole, and a through hole. The head strap unit is connected between the side straps. The intake tube extends into the tube-connecting hole.

### BACKGROUND OF THE INVENTION

1. Field of the Invention

This invention relates to a breathing assistance apparatus, and more particularly to a breathing assistance apparatus that includes two nasal prongs and that is adapted to deliver gases to a patient.

2. Description of the Related Art

Referring to FIGS. 1, 2, and 3, a conventional breathing assistance apparatus 10 includes a unitary nasal mask unit 11 made of silicone gel, two first head straps 12 mounted respectively on two opposite sides of the nasal mask unit 11, a second head strap 13 connected between the first head straps 12, and an intake tube 14 connected to the nasal mask unit 11.

The nasal mask unit 11 includes a main body 15, two connecting lugs 16, and two nasal prongs 17. The main body 15 includes an inner wall 151 abutting against a portion 21 of a face of a user 20 between the nose and the mouth, an outer wall 153 converging outwardly from the inner wall 151 and formed with a tube-connecting hole 152, and a compartment 154 formed between the inner and outer walls 151, 153. The two connecting lugs 16 extend respectively from left and right sides of the inner wall 151 away from each other. The two nasal prongs 17 extend inwardly from the inner wall 151, and are in fluid communication with the compartment 154.

Although the aforesaid breathing assistance apparatus 10 is lightweight, and is made at a relatively low cost, it suffers from the following disadvantages:

1. The two nasal prongs 17 are plugged into the nostrils 21′ of the user 20, and are aligned with the nasal cavity 22. As such, gases can be supplied into the two nasal prongs 17 through the intake tube 14. However, if the two first head straps 12 are pulled inadvertently, they will move to thereby deform the main body 15 and the nasal prongs 17, as shown in FIGS. 4 and 5. Consequently, the nasal prongs 17 are misaligned from the nasal cavity 22, thereby affecting adversely delivery of gases.

2. Since the two nasal prongs 17 are formed integrally with the main body 15, the distance between the two nasal prongs 17 cannot be adjusted. The inter-nostril distances of different users, however, may be different. If a significant difference occurs between the inter-nostril distance of the user and the distance between the two nasal prongs 17, an extreme discomfort feeling will be caused to the user.

### SUMMARY OF THE INVENTION

The object of this invention is to provide a breathing assistance apparatus that can supply effectively gases into the nasal cavity of the user and that can be worn comfortably.

According to an aspect of this invention, there is provided a breathing assistance apparatus adapted to deliver gases to a patient, the breathing assistance apparatus comprising:

a nasal mask unit including a rear wall having an inner surrounding surface defining a prong-receiving hole extending along an axis, a forwardly converging front wall extending forwardly from an outer periphery of the rear wall and formed with a tube-connecting hole therethrough along the axis, an insert groove formed in the inner surrounding surface, and two side straps extending respectively from two opposite sides of the rear wall, the front and rear walls cooperating to define a compartment therebetween that is in fluid communication with the tube-connecting hole and the prong-receiving hole;

an insert plate inserted into the insert groove to thereby be positioned in the prong-receiving hole, the insert plate having two mounting holes;

two nasal prongs each having a nose-engaging end portion, a plate-engaging end portion opposite to the nose-engaging end portion and fitted within a corresponding one of the mounting holes in the insert plate, and a through hole extending through the nose-engaging end portion and the plate-engaging end portion;

a head strap unit connected between the side straps of the nasal mask unit; and

an intake tube extending into the tube-connecting hole in the nasal mask unit.

Since the insert plate and the nasal mask unit are two separate members, when the side straps are pulled or pushed, the insert plate and the two nasal prongs cannot move, so as to promote the gas-supplying effect.

According to another aspect of this invention, there is provided a breathing assistance apparatus adapted to deliver gases to a patient, said breathing assistance apparatus comprising:

a nasal mask unit including a rear wall having an inner surrounding surface defining a prong-receiving hole extending along an axis, a forwardly converging front wall extending forwardly from an outer periphery of said rear wall and formed with a tube-connecting hole therethrough along the axis, an insert groove formed in said inner surrounding surface, and two side straps extending respectively from two opposite sides of said rear wall, said front and rear walls cooperating to define a compartment therebetween that is in fluid communication with said tube-connecting hole and said prong-receiving hole;

an insert plate unit including a plurality of insert plates, a selected one of which is inserted into said insert groove to thereby be positioned in said prong-receiving hole, each of said insert plates having two mounting holes spaced apart from each other by an inter-hole distance, the inter-hole distances of said insert plates being different from each other;

two nasal prongs each having a nose-engaging end portion, a plate-engaging end portion opposite to said nose-engaging end portion and fitted within a corresponding one of said mounting holes in said insert plate, and a through hole extending through said nose-engaging end portion and said plate-engaging end portion;

a head strap unit connected between said side straps of said nasal mask unit; and

an intake tube extending into said tube-connecting hole in said nasal mask unit.

Since the inter-hole distances of the insert plates are different from each other, the user can elect one of the insert plates conforming to his or her inter-nostril distance, thereby resulting in a comfort feeling to the user.

### BRIEF DESCRIPTION OF THE DRAWINGS

These and other features and advantages of this invention will become apparent in the following detailed description of the preferred embodiments of this invention, with reference to the accompanying drawings, in which:

FIG. 1 is an exploded perspective view of a conventional breathing assistance apparatus;

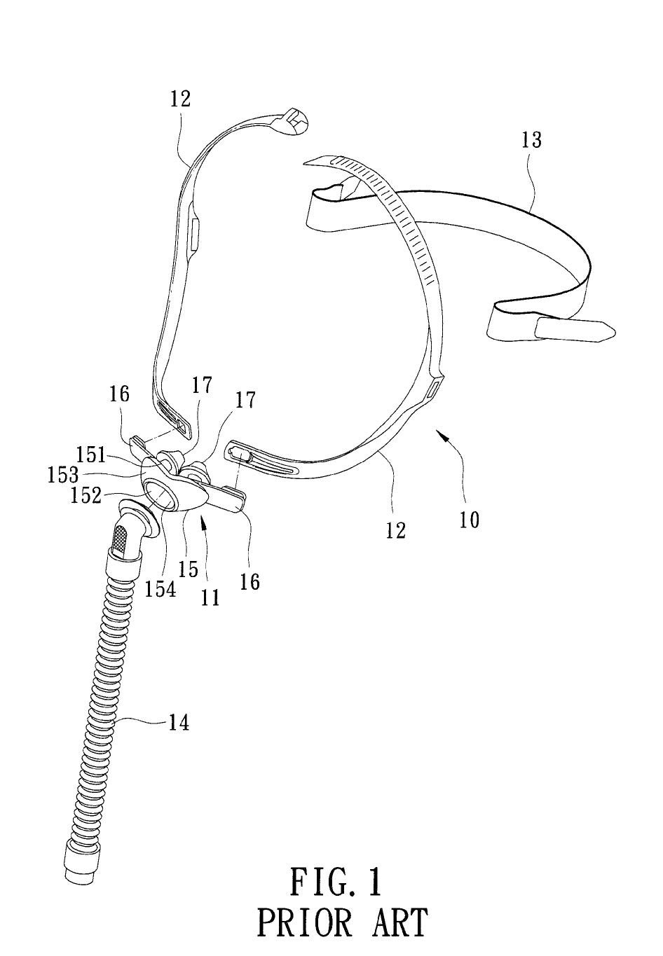

FIG. 2 is a fragmentary side view of the conventional breathing assistance apparatus, illustrating how it is worn by a user;

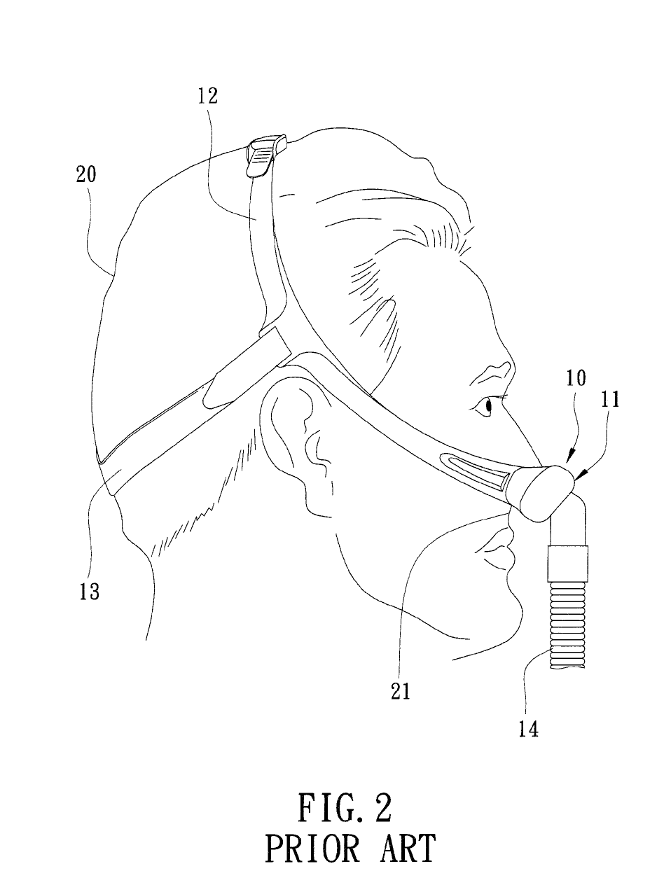

FIG. 3 is a schematic view illustrating that a nasal prong of the conventional breathing assistance apparatus is aligned with a nasal cavity of the user;

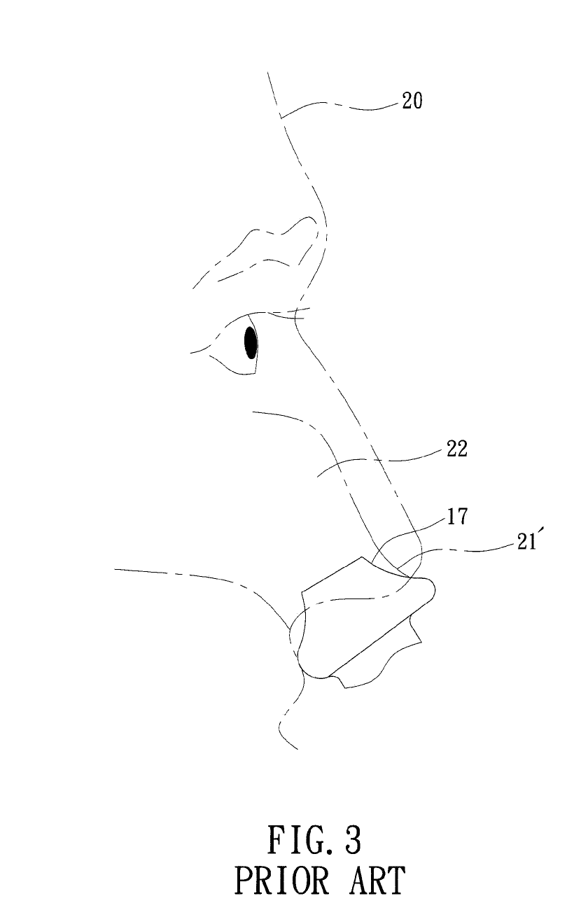

FIG. 4 is a fragmentary schematic top view of the conventional breathing assistance apparatus, illustrating accidental movement of two nasal prongs;

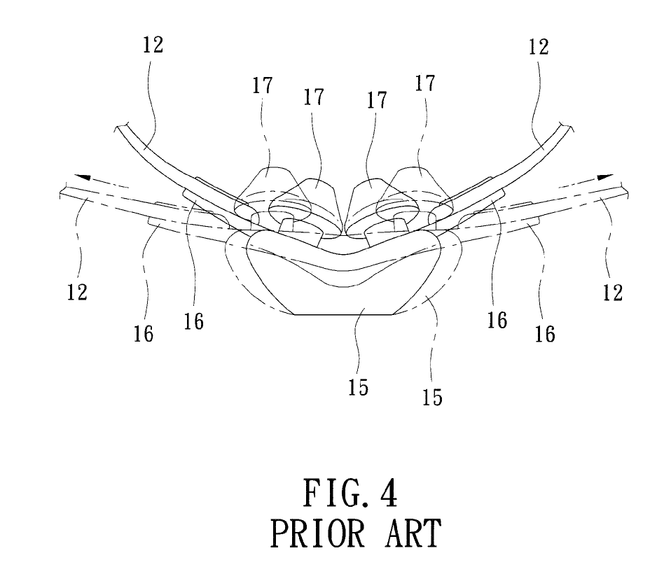

FIG. 5 is a view similar to FIG. 3 but illustrating the nasal prong is misaligned from the nostril cavity;

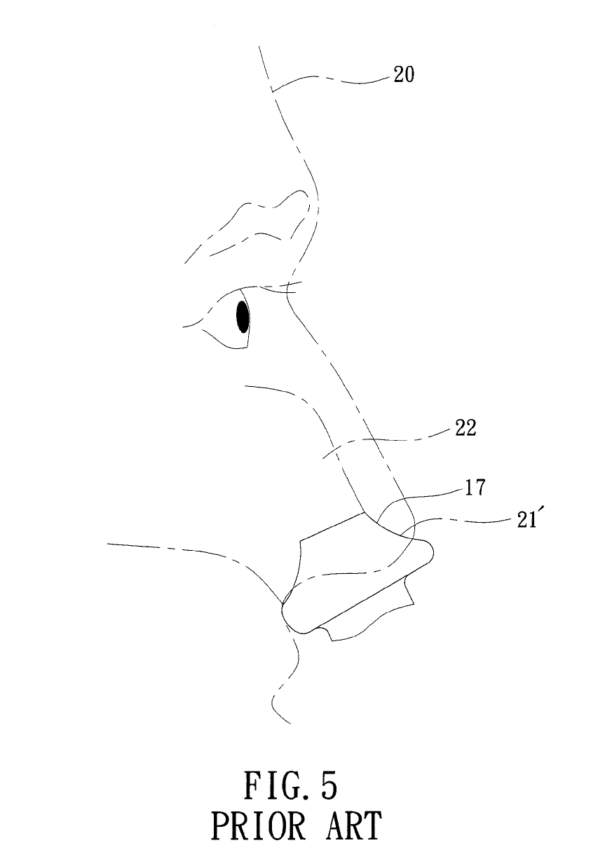

FIG. 6 is an exploded perspective view of the first preferred embodiment of a breathing assistance apparatus according to this invention;

FIG. 7 is an assembled perspective view of the first preferred embodiment;

FIG. 8 is a fragmentary, partly sectional view of the first preferred embodiment, illustrating a nasal mask unit, an insert plate, and a nasal prong;

FIG. 9 is a schematic front view of the first preferred embodiment in a used state;

FIG. 10 is a schematic side view of the first preferred embodiment in the used state;

FIG. 11 is a schematic side view illustrating how a nasal prong is plugged into a nostril of a user to align with a nasal cavity of the user;

FIG. 12 is a fragmentary top view of the first preferred embodiment, illustrating that two side straps are pulled outwardly;

FIG. 13 is an exploded perspective view of the second preferred embodiment of a breathing assistance apparatus according to this invention; and

FIG. 14 is a schematic front view of three insert plates of the second preferred embodiment, which have different inter-hole distances.

### DETAILED DESCRIPTION OF THE PREFERRED EMBODIMENTS

Before the present invention is described in greater detail in connection with the preferred embodiments, it should be noted that similar elements and structures are designated by like reference numerals throughout the entire disclosure.

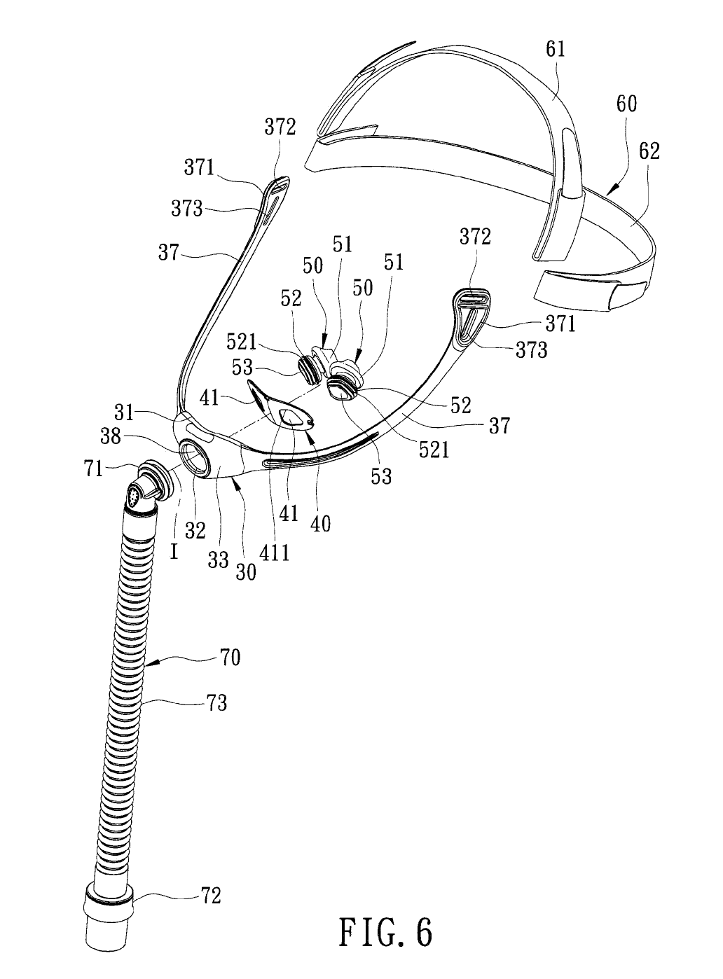

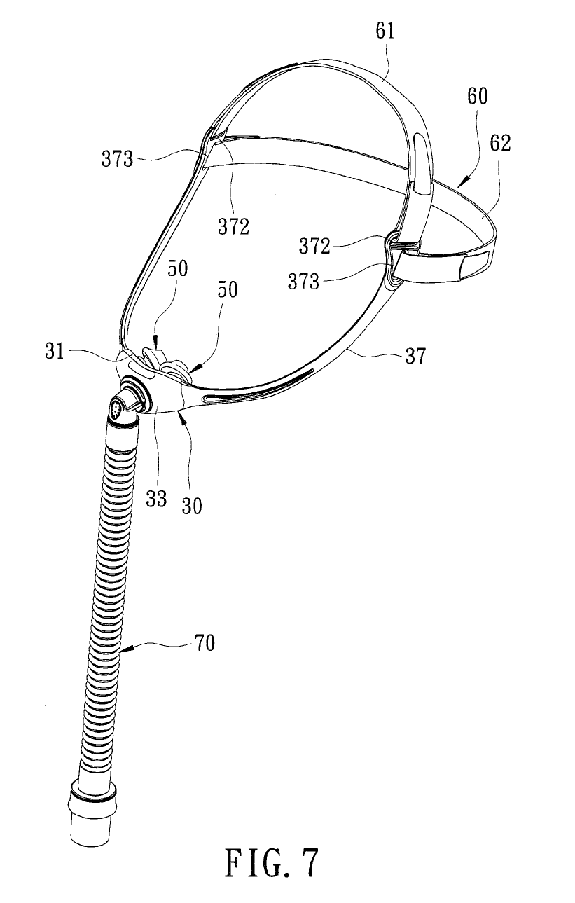

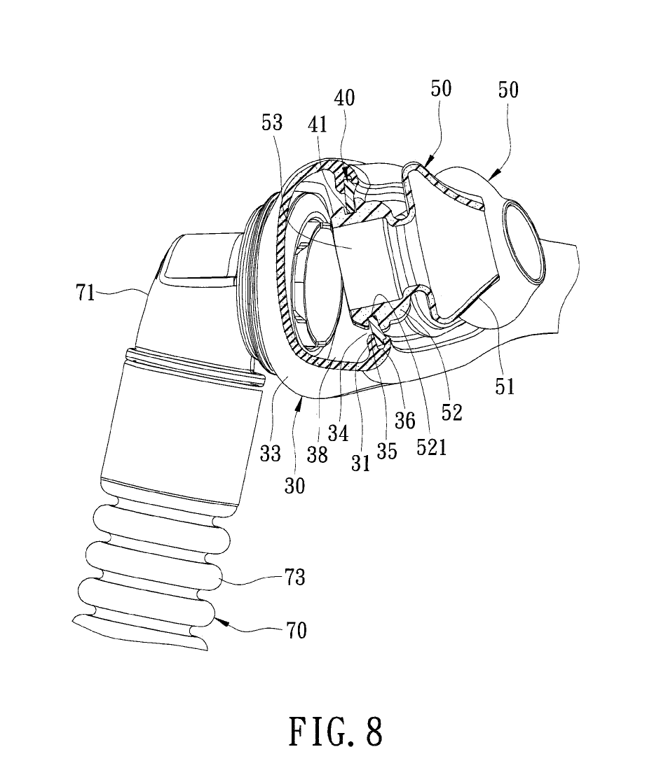

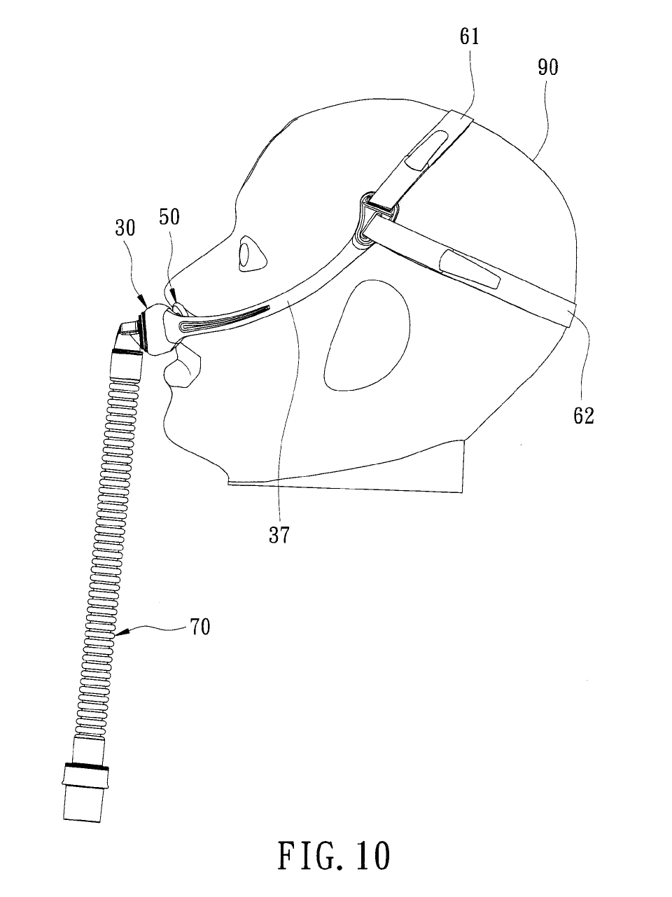

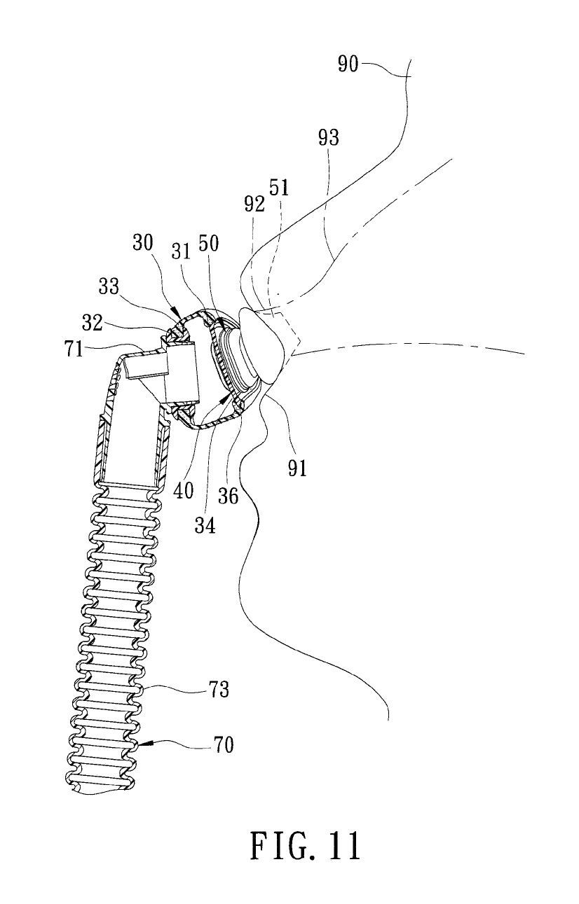

Referring to FIGS. 6, 7, 8, 9, 10, and 11, the first preferred embodiment of a breathing assistance apparatus according to this invention is adapted to deliver gases to a user 90, such as patient. The breathing assistance apparatus includes a unitary nasal mask unit 30, an insert plate 40, two nasal prongs 50, a head strap unit 60, and an intake tube 70.

The nasal mask unit 30 includes a rear wall 31 adapted to abut against a portion 91 of a face of an user 90 between the nose and the mouth, and a forwardly converging front wall 33 extending forwardly from an outer periphery of the rear wall 31 and formed with a tube-connecting hole 32 extending therethrough along an axis (I). The rear wall 31 has an inner surrounding surface 35 defining a prong-receiving hole 34 extending along the axis (I). The nasal mask unit 30 further includes an insert groove 36 formed in the inner surrounding surface 35, and two side straps 37 extending respectively from two opposite sides of the rear wall 31. The front and rear walls 33, 31 cooperate to define a compartment 38 therebetween that is in fluid communication with the tube-connecting hole 32 and the prong-receiving hole 34.

Each of the side straps 37 has a pair of first and second penetration holes 372, 373.

Preferably, the nasal mask unit 30 is made of a flexible material, such as silicone gel, to provide a comfort feeling to the user 90.

The insert plate 40 is inserted into the insert groove 36 to thereby be positioned in the prong-receiving hole 34, and has two mounting holes 41 located respectively at left and right sides of the axis (I).

The nasal prongs 50 are made of silicone gel. Each of the nasal prongs 50 has a nose-engaging end portion 51 plugged into the corresponding nostril of the user 90, a plate-engaging end portion 52 opposite to the nose-engaging end portion 51 and fitted within the corresponding mounting hole 41 in the insert plate 40, and a through hole 53 extending through the nose-engaging end portion 51 and the plate-engaging end portion 52.

The nose-engaging end portions 51 of the nasal prongs 50 are frustoconical.

The plate-engaging end portion 52 of each of the nasal prongs 50 has an annular outer surface formed with an annular retaining groove 521. Each of the mounting holes 41 in the insert plate 40 is defined by a hole-defining wall 411, which is engaged within the annular retaining groove 521 of the corresponding nasal prong 50. As such, removal of the plate-engaging end portions 52 of the nasal prongs 50 from the insert plate 40 is prevented, and the through hole 53 is in fluid communication with the compartment 38.

The head strap unit 60 is connected between the side straps 37, and includes a first head strap 61 extending through the first penetration holes 372 in the side straps 37 and around the vertex of the user 90, and a second head strap 62 extending through the second penetration holes 373 in the side straps 37 and around the hindbrain of the user 90 and disposed below the first head strap 61.

The intake tube 70 includes a first joint 71 extending into the tube-connecting hole 32 in the nasal mask unit 30, a second joint 72 opposite to the first joint 71 and connected to a gas supply device (not shown), and a connecting tube 73 connected between the first and second joints 71, 72.

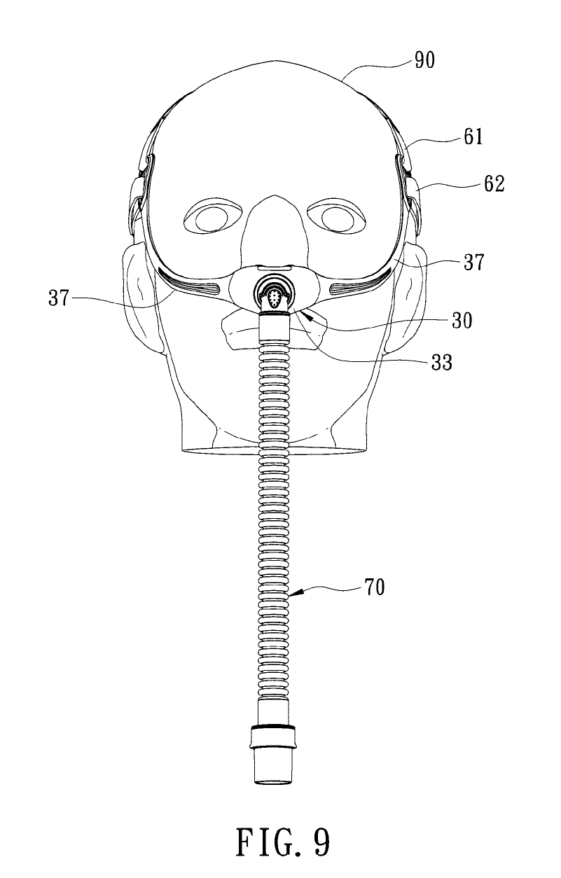

With particular reference to FIGS. 9, 10, and 11, during use, the nose-engaging end portions 51 of the nasal prongs 50 are first plugged into the nostrils 92 of the user 90, respectively, an are aligned with the nasal cavity 93 of the user 90. Next, the first head straps 61 are placed over the vertex of the user 90, and are adjusted to appropriate lengths, and the second head straps 62 are placed over the hindbrain of the user 90, and are adjusted to appropriate lengths, thereby finishing the wearing operation.

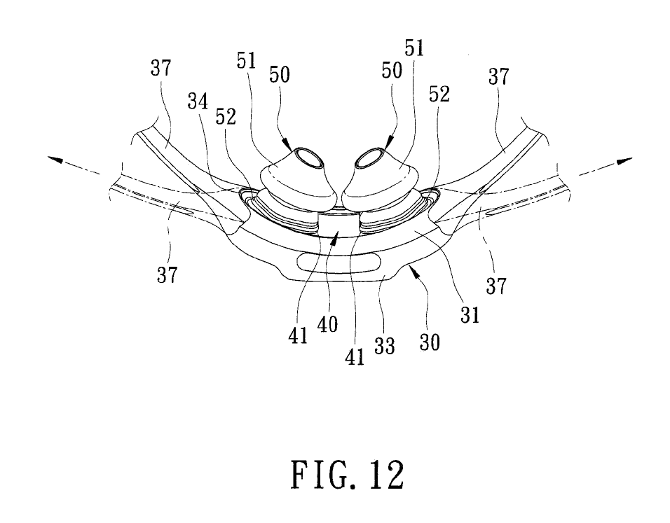

With particular reference to FIGS. 6, 8, 11, and 12, since the insert plate 40 is inserted into the insert groove 36 in the nasal mask unit 30 to thereby be positioned within the prong-receiving hole 34, and since the insert plate 40 and the nasal mask unit 30 are two separate members, if the two side straps 37 of the nasal mask unit 30 are pulled outwardly from the positions shown by the solid lines in FIG. 12 to the positions shown by the phantom lines in FIG. 12, the insert plate 40 cannot be deformed and moved. As a consequence, the nasal prongs 50 remain in situ to thereby be still aligned with the nasal cavity 93 of the user 90, so that the gas supplying effect is improved.

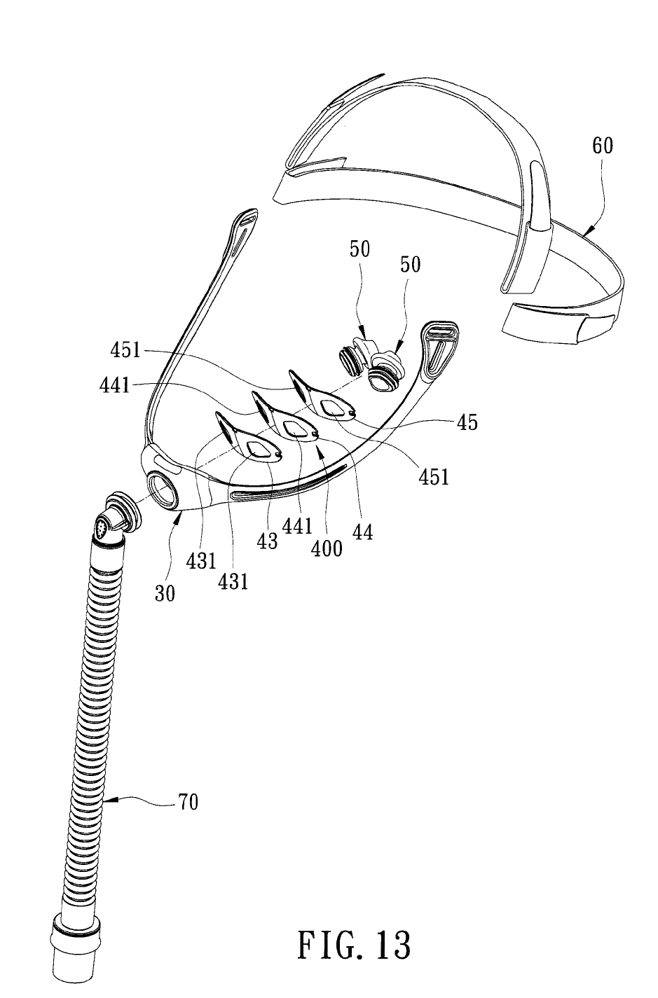

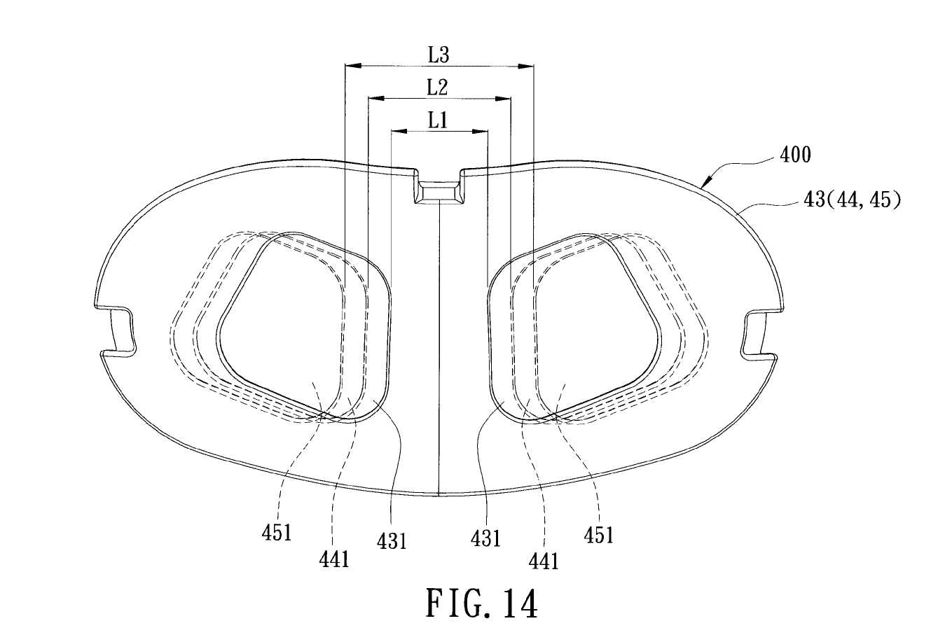

FIGS. 13 and 14 show the second preferred embodiment of a breathing assistance apparatus according to this invention, which is similar in construction to the first preferred embodiment except that the insert plate 40 (see FIG. 6) is replaced with an insert plate unit 400.

The insert plate unit 400 includes first, second, and third insert plates 43, 44, 45 each formed with two mounting holes 431, 441, 451 spaced apart from each other by an inter-hole distance (L1, L2, L3). The inter-hole distances (L1, L2, L3) of the first, second, and third insert plates 43, 44, 45 are different from each other. In this embodiment, L2 is greater than L1, and smaller than L3.

The user 90 can elect one of the first, second, and third insert plates 43, 44, 45 conforming to the inter-nostril distance of the user 90 to connect with the nasal mask unit 30, thereby resulting in an extreme comfort feeling to the user 90.

In view of the above, the breathing assistance apparatus of this invention has the following advantages:

1. Movement of the nasal prongs 50 resulting from accidental operation of the side straps 37 can be prevented to allow for effective delivery of gases.

2. A selected one of the first, second, and third insert plates 43, 44, 45 can be connected with the nasal mask unit 30 to promote the user comfort.

3. The two nasal prongs 50 can be replaced to allow for continued use of the breathing assistance apparatus by the same user 90, thereby meeting cost-effective and environmental protection requirement.

With this invention thus explained, it is apparent that numerous modifications and variations can be made without departing from the scope and spirit of this invention. It is therefore intended that this invention be limited only as indicated by the appended claims.

I claim:

1. A breathing assistance apparatus adapted to deliver gases to a patient, said breathing assistance apparatus comprising:
a nasal mask unit including a rear wall having an inner surrounding surface defining a prong-receiving hole extending along an axis, a forwardly converging front wall extending forwardly from an outer periphery of said rear wall and formed with a tube-connecting hole therethrough along the axis, an insert groove formed in said inner surrounding surface, and two side straps extending respectively from two opposite sides of said rear wall, said front and rear walls cooperating to define a compartment therebetween that is in fluid communication with said tube-connecting hole and said prong-receiving hole;
an insert plate inserted into said insert groove to thereby be positioned in said prong-receiving hole, said insert plate having two mounting holes;
two nasal prongs each having a nose-engaging end portion, a plate-engaging end portion opposite to said nose-engaging end portion and fitted within a corresponding one of said mounting holes in said insert plate, and a through hole extending through said nose-engaging end portion and said plate-engaging end portion;
a head strap unit connected between said side straps of said nasal mask unit; and
an intake tube extending into said tube-connecting hole in said nasal mask unit.

2. The breathing assistance apparatus as claimed in claim 1, wherein each of said side straps of said nasal mask unit has a pair of first and second penetration holes, said head strap unit including a first head strap extending through said first penetration holes in said side straps, and a second head strap extending through said second penetration holes in said side straps.

3. The breathing assistance apparatus as claimed in claim 1, wherein said plate-engaging end portion of each of said nasal prongs has an annular outer surface formed with an annular retaining groove, each of said mounting holes in said insert plate being defined by a hole-defining wall, which is engaged within said annular retaining groove in a corresponding one of said nasal prongs.

4. The breathing assistance apparatus as claimed in claim 1, wherein said nasal mask unit and said nasal prongs are made of silicone gel.

5. A breathing assistance apparatus adapted to deliver gases to a patient, said breathing assistance apparatus comprising:
a nasal mask unit including a rear wall having an inner surrounding surface defining a prong-receiving hole extending along an axis, a forwardly converging front wall extending forwardly from an outer periphery of said rear wall and formed with a tube-connecting hole therethrough along the axis, an insert groove formed in said inner surrounding surface, and two side straps extending respectively from two opposite sides of said rear wall, said front and rear walls cooperating to define a compartment therebetween that is in fluid communication with said tube-connecting hole and said prong-receiving hole;
an insert plate unit including a plurality of insert plates, a selected one of which is inserted into said insert groove to thereby be positioned in said prong-receiving hole, each of said insert plates having two mounting holes spaced apart from each other by an inter-hole distance, the inter-hole distances of said insert plates being different from each other;
two nasal prongs each having a nose-engaging end portion, a plate-engaging end portion opposite to said nose-engaging end portion and fitted within a corresponding one of said mounting holes in said insert plate, and a through hole extending through said nose-engaging end portion and said plate-engaging end portion;
a head strap unit connected between said side straps of said nasal mask unit; and
an intake tube extending into said tube-connecting hole in said nasal mask unit.

6. The breathing assistance apparatus as claimed in claim 5, wherein each of said side straps of said nasal mask unit has a pair of first and second penetration holes, said head strap unit including a first head strap extending through said first penetration holes in said side straps, and a second head strap extending through said second penetration holes in said side straps.

7. The breathing assistance apparatus as claimed in claim 5, wherein said plate-engaging end portion of each of said nasal prongs has an annular outer surface formed with an annular retaining groove, each of said mounting holes in said first, second, third insert plates being defined by a hole-defining wall, which is engageable within said annular retaining groove in a corresponding one of said nasal prongs.

8. The breathing assistance apparatus as claimed in claim 5, wherein said nasal mask unit and said nasal prongs are made of silicone gel.
